(19) United States
(12) Patent Application Publication
(10) Pub. No.: US 2005/0209328 A1
(43) Pub. Date: Sep. 22, 2005

(54) Alphahydroxyacids with ultra-low metal concentration

(76) Inventors: Charles Clinton Allgood, Elkton, MD (US); Stephen Thomas Breske, Wilmington, DE (US); Michael Thomas Sheehan, Corpus Christi, TX (US); Michael Harry Willard, Dunbar, WV (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/069,877

(22) Filed: Feb. 28, 2005

Related U.S. Application Data
(60) Provisional application No. 60/554,754, filed on Mar. 19, 2004.

(51) Int. Cl. A61K 31/19

(52) U.S. Cl. 514/557; 562/567; 562/568

(57) ABSTRACT

A composition and a process for producing the composition are disclosed. The composition comprises an alphahydroxyacid and one or more metals in which the metal is present in lower than about 1,000 μg/kg of the composition. The process comprising contacting an acidic ion exchange resin with an aqueous composition comprising a soluble alphahydroxyacid and a total metal concentration, individual metal concentration, or both, higher than that desired to produce a resin-treated alphahydroxyacid solution having reduced total metal concentration. Also disclosed is a process that can be used for cleaning or removing residues from semiconductor substrates and/or equipment by using a solution, which comprises an alphahydroxyacid.

### FIELD OF THE INVENTION

The invention relates to a composition comprising an alphahydroxyacid having low total metal concentration and to processes therefor and therewith.

### BACKGROUND OF THE INVENTION

The manufacture of advanced electronic devices such as semiconductor components historically has used thin film deposition and etching processes to construct three-dimensional circuits, typically using aluminum conductors and silica (SiO₂) insulation layers. Connections between layers are constructed using optical lithography, photoresist patterning and plasma etching to create a complex and extremely small-scale pattern of connecting holes through the silica insulating layers. Several hundred steps may be required for the manufacture of some semiconductor chips, with exacting requirements at each step. The constant need for increased device performance along with micro-miniaturization is presently leading to a switch to copper conductors and better insulating (low-k dielectric) films such as doped silica, fluorinated or porous insulation layers. For the Al/SiO₂ systems, post-etch cleaning formulations relied on formulations containing such chemicals as hydroxylamine or other solvents. These formulations, however, do not meet the requirements of the newer advanced chip designs and materials of construction.

In addition, there is a desire in the industry to move away from solvents to more environmentally friendly aqueous-based cleaners. For instance, a dilute aqueous alphahydroxyacid (hereinafter “AHA”) such as glycolic acid is known to work well in the cleaning of copper in printed wiring boards; a relatively crude process compared with the manufacture of modern semiconductor chips. Merck Index, 12^{th} Edition, 1996, (Merck & Co., Inc., Whitehouse Station, N.J., p. 4507), shows glycolic acid uses include “copper brightening, decontamination cleaning, . . . pickling, cleaning, and chemical milling of metals.” In modern semiconductor chips, features such as conducting “via” or holes, are of the order of 60 nm in diameter. Another requirement during the many stages of the construction of a semiconductor chip is that the levels of metals, particularly metal ions in cleaning formulations must be limited to concentrations at the microg/kg (μg/kg, parts per billion, or ppb) level. Residual metal contamination left in the holes can result in unwanted conductive pathways or alter the composition and, therefore, the electrical performance of various film layers, resulting in a diminished yield of micro-assemblies meeting the rigid final performance specifications. In semiconductor wafer manufacture, there is a need for ultra low metallic impurities (μg/kg levels) for any processing material or liquid that will contact the wafer in order to avoid affecting the electrical properties of the integrated circuits being produced.

An AHA, such as glycolic acid, can be produced by a number of routes such as, for instance, a strong acid-catalyzed reaction of carbon monoxide, formaldehyde, and water optionally using sulfuric acid as the catalyst, depicted as:
CO+HCHO+H₂O→HOCH₂COOH

This carbonylation process is well known and is disclosed in U.S. Pat. Nos. 2,135,064; 2,152,852; and 2,037,654 as well as in U.S. Pat. No. 3,859,349 and WO 92/05138. The entire disclosures of these patents are hereby incorporated by reference. Aqueous solutions of glycolic acid are made up of mixtures of monomeric glycolic acid and soluble polyacids (predominantly hydroxyacetic acid dimer) in equilibrium, the ratio being determined by solution concentration. The polyacids can be hydrolyzed upon dilution of 70% glycolic acid with water to 20% by weight or less, and refluxing.

Other processes include chloroacetic acid hydrolysis and fermentation processes.

These processes produce crude glycolic acid that is preferably purified prior to use or sale.

The commercial grades of glycolic acid are typically 70% solutions of the acid in water. For conventional uses, the concentration of various metal cations, including sodium, magnesium, aluminum, and potassium, are acceptable. For instance, the United States Pharmacopoeia (USP) specification for glycolic acid limits arsenic to 3 mg/kg (3 parts per million or ppm, 3000 μg/kg), heavy metals to 0.001% (10 mg/kg, 10000 μg/kg), but the limitation for metals such as sodium and potassium are only included to the extent that the residue on ignition shall be not more than 0.05% (500 mg/kg, 500000 μg/kg). That is, there is no specific requirement on sodium and potassium. Analyses of the typical total metal cation concentration of commercial glycolic acid is about 20-35 mg/kg (20000-35000 μg/kg) total analyzed metals, with individual metals ranging from <1 to about 20 mg/kg (<1000 to 20000 μg/kg). These concentrations of metal contaminants are orders of magnitude too high for satisfactory use in semiconductor cleaning or surface preparation applications, for example in post-etch cleaning formulations. The required specification for an organic acid to be used as cleaning or surface preparation agents in modern semiconductor applications is at least 100-fold less. For semiconductor use, it is anticipated that specifications of 50-100 μg/kg are needed, and preferably of the order of 10 μg/kg. A considerable number of metals are included in the ultra-low metal specification. Sodium and potassium ions can be the most abundant and, as monovalent ions, also the most difficult to minimize.

An AHA such as glycolic acid is nonvolatile and cannot be distilled even under reduced pressure. Heating molten glycolic acid can produce poly(hydroxyacetic acid), termed polyglycolide, and water via a self-esterification reaction. Purification of glycolic acid from metal cations by distillation is therefore impractical.

Another specific AHA of interest in the practice of the present invention is tartaric acid (2,3-dihydroxybutanedioic acid), typically in the L- or DL-isomeric forms. Again, the USP/Food and Chemicals Codex specification for food grade L-tartaric acid has <0.001% (10 ppm, 10000 μg/kg) heavy metals, a residue on ignition of not more than 0.1% (10000 ppm, 10⁷ μg/kg), and purity not less than 97.7%, similar to those for glycolic acid. Thus, food grade L-tartaric acid has a permissible total metal concentration far higher than that desired for satisfactory semiconductor cleaning or surface preparation applications.

Various sequences of purification have been disclosed such as those disclosed in U.S. Pat. No. 3,859,349. The disclosure in U.S. Pat. No. 3,859,349 shows reduction of iron to 10 mg/kg (10000 μg/kg) maximum and copper to 5 mg/kg (5000 μg/kg) maximum. Similarly, WO 92/05138 discloses reduction of iron to 2.6 mg/kg (2600 μg/kg).

It would be desirable to produce an AHA, including glycolic acid, with extremely low metal concentration as an ingredient in post-etch cleaners. The present invention provides AHAs with the required extremely low metal concentration.

### SUMMARY OF THE INVENTION

The present invention provides a composition comprising an alphahydroxyacid and one or more metals wherein the total metal concentration is less than 1000 μg/kg. The present invention further provides a composition comprising an alphahydroxyacid and one or more metals wherein the concentration of any individual metal of the composition is less than 250 μg/kg. In one embodiment of this invention, the metals are selected from the group consisting of aluminum, calcium, chromium, copper, iron, lead, magnesium, manganese, nickel, potassium, sodium, and zinc and combinations of two or more thereof. Preferably, the composition is in the form of a solution, more preferably, an aqueous solution.

The concentration of alphahydroxyacid in a solution composition of this invention can be as low as 0.01%, based on the total weight of the composition and as high as the solubility limit of the acid in the solution. Preferably, the concentration of the alphahydroxyacid is less than the solubility limit to avoid precipitation and/or crystallization of the alphahydroxyacid. Desirable ranges of concentration of the alphahydroxyacid in a solution composition of this invention are from 50% to 99% of the solubility limit of the acid in the solution, and preferably 75% to 98% of the solubility limit of the acid in the solution.

The composition of this invention may be produced by a process of this invention. This process comprises contacting an aqueous composition which comprises an alphahydroxyacid and one or more metals selected from the group consisting of wherein the total metal concentration is greater than 1000 μg/kg with a strongly acidic cation resin under conditions effective to reduce the total metal concentration to less than 1000 μg/kg.

The present invention further provides a process comprising contacting a substrate with a composition comprising an alphahydroxyacid and one or more metals selected from the group consisting of wherein the total metal concentration is less than 1000 μg/kg. The substrate can be a surface or structure of a fully or partially fabricated electronic device or of processing equipment composed of insulating and/or non-insulating materials, and combinations of two or more thereof. The materials may be, for example, but not limited to, silicon, silicon dioxide, aluminum, copper, or tungsten or composites thereof.

### DETAILED DESCRIPTION OF THE INVENTION

Trademarks herein are shown in upper case.

The term “total metal concentration” as used herein means the total metal concentration of the specified metals as analyzed, and includes ionic and nonionic forms. The term “individual metal concentration” as used herein means the metal concentration of that individual metal as analyzed, and includes ionic and nonionic forms.

The terms “deionized water” or “DI water” as used herein means purified water having a resistivity of >15 M ohm and preferably >17 M ohm. Resistivity measurements utilize a conductivity/resistivity probe, such as a NIST-traceable Digital Conductivity Meter, No. 23226-501, from made by VWR International (West Chester, Pa., USA). DI water suitable for the practice of the present invention is often obtained from “turn-key” units such as a Sybron-Barnstead “NANOPURE II” unit, available from Barnstead-Thermolyne (Dubuque, Iowa, USA).

The invention comprises a composition, which comprises an AHA and one or more metals in which the total metal concentration is less than about 1000 and preferably less than about 500 μg/kg of the composition. Individual metal concentrations are less than about 250, preferably less than about 150, and more preferably less than about 100 μg/kg of the composition.

In one particular composition, the metal is selected from the group consisting of sodium, magnesium, aluminum, potassium, calcium, iron, nickel and zinc and combinations of two or more thereof. This composition comprises an alphahydroxyacid having a concentration of sodium, magnesium, aluminum, potassium, calcium, iron, nickel, and zinc of less than 200 and preferably less than 100 μg/g of the composition. In the various applications of the compositions of the present invention, the specifications for total metals and for individual metals are expected to vary. For instance, for use in a copper-based system, specifications for the reduction of the copper concentration would be substantially less stringent.

Such low total metal concentration alphahydroxyacids, that is, AHAs comprising one or more metals wherein the total metal concentration is less than about 1000 μg/kg and wherein individual metal concentrations are less than about 250 μg/kg, are referred to herein as “electronics grade” or “semiconductor grade” wet chemicals, suitable as components of a number of cleaning and surface preparation chemicals, for instance as components of post-etch cleaning formulations. The actual total metal concentration and concentrations of individual metals varies, depending on the end use of the alphahydroxyacid composition. Therefore, for certain applications, “electronics grade” or “semiconductor grade” alphahydroxyacid may require a total metal concentration of less than 500 μg/kg and an individual metal concentration of less than 100 μg/kg. Such formulations enable the reliable cleaning of etched “via” or holes.

In another embodiment, the invention comprises a composition, which comprises glycolic acid and one or more metals in which the total metal concentration is less than about 200, preferably less than about 150 and more preferably less than about 100 μg/kg of the composition. Individual metal concentrations are less than about 100, preferably less than about 50, and more preferably less than about 25 μg/kg of the composition.

Generally all known water-soluble alphahydroxyacids (AHA) can be suitable for use in the composition and process of the present invention. Of particular interest are those AHAs useful in the semiconductor industry such as, for example, those selected from the group consisting of glycolic acid (alphahydroxyacetic acid), lactic acid (alphahydroxypropanoic acid), tartaric acid (2,3-dihydroxybutanedioic acid), typically in the L- or DL-isomeric forms, and citric acid (2-hydroxy-1,2,3-propanetricarboxylic acid). Preferably the AHA is glycolic acid or tartaric acid. More preferably, the AHA is glycolic acid.

The AHA composition of this invention comprises one or more metals selected from the group consisting of aluminum, calcium, chromium, copper, iron, lead, magnesium, manganese, nickel, potassium, sodium, and zinc. The metals in the total metal concentration may include other metals as well. However end use applications of the composition may prescribe particular maximum concentrations of these other metals. The preceding list of metals is alphabetical and not in order of preponderance or importance. It is common in the electronics industry to require that the chemicals they use meet low concentration limits for these metals.

Another important factor is a propensity for chromium to exist predominately as the cation and to coexist as a Cr(VI) anion, in the form of the chromate/dichromate equilibrium shown below. For this reason (among others?), Cr(VI) has proven to be particularly difficult to reduce to a concentration of less than about 250 μg/kg in an alphahydroxyacid. In the anionic form the chromium passes freely through a cationic resin bed but nevertheless appears as chromium in effluent analyses.
Cr₂O₇²⁻+2OH⁻ [symbol] 2CrO₄²⁻+H₂O
Alkaline medium:dichromate [symbol] acid medium:chromate

Analytical methods for the measurement of trace amounts of Cr(VI) in water are well known since Cr(VI) is a notorious environmental pollutant and is much more toxic than the trivalent Cr(III) cation. However, techniques for the same analysis in a concentrated solution of an AHA were not available. In cases where Cr(VI) anions are present, removal can be effected by the use of an anion exchange resin or by prior reduction. In the presence of an AHA, a thoroughly-washed anionic resin in the hydroxide form will rapidly form the AHA form, but since the alphahydroxy anion is much weaker than the Cr(VI) anion, the resin is still effective at removing the Cr(VI) anion. The anionic resin may be in a separate or layered bed with the cation resin, or, preferably in a mixed bed. Methods for regenerating layered and mixed bed resins are well known to those skilled in the art.

Alternatively, the Cr(VI) anion is may be reduced to the chromium cation, Cr(III), by any suitable method. According to any of these methods, the process of the invention, comprises, in a step prior to providing one or more vessels comprising therein a cation resin, as described in detail hereinbelow, a step of treating an AHA to reduce Cr(VI) compounds to Cr(III) compounds. These methods include, for example, contacting an AHA with a reductant. The reductant can be selected from the group consisting of a solution comprising a soluble reducing agent or a gaseous reductant, such as sulfur dioxide (which forms sulfurous acid in solution). The soluble reducing agent can be selected from the group consisting of a ferrous salt, hydrogen peroxide, potassium iodide, and sodium sulfite. The use of a small stoichiometric excess of a reducing solution, such as, for example, a solution of ferrous sulfate, effects the conversion of Cr(VI) to Cr(III). However, use of metal salts disadvantageously add both cations and anions when the overall objective of the process is to minimize cations. Methods using a reductant that eliminate or minimize addition of other cations may be used, such as contacting an AHA with gaseous sulfur dioxide (forming sulfurous acid in solution), or hydrogen peroxide, as shown below. Other reducing agents that effect the reduction are well known to those skilled in the art. Ferrous ion reduction:
Cr₂O₇²⁻+14H⁺+6Fe²⁺ [symbol] 2Cr³⁺+6Fe³⁺+7H₂O

Sulfur dioxide reduction:
SO₂+H₂O [symbol] 2H⁺+SO₃²⁻
CrO₇²⁻+8H⁺+3SO₃²⁻ [symbol] 2Cr³⁺+4H₂O+3SO₄²⁻

Hydrogen peroxide reduction:
CrO₇²⁻+8H⁺+3H₂O₂ [symbol] 2Cr³⁺+7H₂O+3O₂

The amount of reductant should be carefully calculated and is very small, at least a stoichiometric amount and typically should be about 2 to about 5 times the stoichiometric amount, calculated assuming all the chromium appearing in the eluate is in the form of Cr(VI).

The composition is generally in the form of a solution, preferably an aqueous solution. The concentration of the alphahydroxyacid in a solution composition can range 0.01%, based on the total weight of the composition up to the solubility limit of the acid in the solution. Preferably, the concentration of alphahydroxyacid is less than the solubility limit to avoid precipitation and/or crystallization of the alphahydroxyacid. Desirable ranges of concentration of the alphahydroxyacid in a solution composition of this invention are from 50% to 99% of the solubility limit of the acid in the solution, and preferably 75% to 98% of the solubility limit of the acid in the solution.

The process comprises (a) providing one or more vessels comprising therein at least one strongly acidic cation resin; (b) contacting the resin with a flow of a strong acid to produce an acid-treated resin; (c) washing the resin with a flow in a concurrent flow direction to the flow of strong acid of deionized water to produce a resin substantially free of soluble acid; (d) contacting the acid-treated and washed resin with a flow in a countercurrent flow direction to the flow of strong acid of a feed composition comprising an alphahydroxyacid and one or more metals wherein the total metal concentration is greater than about 1000 μg/kg and the individual metal concentration is greater than about 250 μg/kg to produce a resin-treated alphahydroxyacid composition and spent resin; and (e) separating and recovering the resin-treated alphahydroxyacid composition.

Optionally and preferably, the AHA feed composition is kept under a blanket of an inert gas, such as nitrogen or any other gas inert under the conditions. Further, optionally, the process further comprises contacting the resin with a flow of deionized water prior to step (b) of contacting the resin with a strong acid, to produce a washed resin. Preferably, the flow of deionized water in this optional step is concurrent with the flow of strong acid. In practice, washing with DI water either before or after contacting the resin with strong acid is continued, until the resistivity of the output is at least about 5 M ohm.

Still further, optionally, the process comprises regenerating the spent resin for reuse after step (e).

The washed resin is optionally mostly or fully hydrated with water. The DI water used is the same as that disclosed above and can be “super DI water” (i.e., 18.3 M ohm).

Counter-current flows of the strong acid and the subsequent AHA solution ensure that the last traces of cations will tend to be at the input of the vessel for the AHA feed composition, maximizing cation removal by minimizing leaching of these last traces of cations into the AHA feed composition. The preferred flow directions are upflow for the strong acid and downflow for the denser AHA feed composition. The reverse, that is, upflow of the denser AHA feed composition, can cause the resin to undesirably expand.

Suitable strongly acidic cation resins include, but are not limited to, sulfonic acid-substituted resins. Specific examples of such strongly acidic cation resins are DOWEX M-31 and DOWEX 650C UPW (Dow Chemical, Midland Mich.), Amberlyst 15 (Rohm & Haas Co., Philadelphia Pa.), and DIAION PKT228L and DIAION SKT20L (Itochu Specialty Chemicals Inc., Japan). The DOWEX and AMBERLYST resins are all sulfonated copolymers of styrene and divinylbenzene, H form, but may differ in the degree of cross-linking and pore size. The DIAION resins are also sulfonated copolymers. A strongly acidic cation resin has strong acid functional groups, i.e., the functional groups are highly dissociated when wet in the pH range 0-14.

Procedures for handling strongly acidic cation resins, DI water, and low total metal concentration solutions are well known to those skilled in the art. Suitable materials of construction for wettable surfaces contacting the alphahydroxyacids with low total metal concentration concentrations are nonmetallic. Example nonmetallic materials suitable as materials of construction or equipment linings include, but are not limited to, perfluorocarbon resins, high density poly(ethylene) (HDPE), high density poly(propylene) (HDPP), polyamides, polyesters, polyimides, polyurethanes, and the like. It cannot be emphasized too strongly that the handling of solutions having extremely low cation concentrations requires rigorous procedures, such as the use of a Class 100 cleanroom environment.

Suitable ion exchange resin vessels are preferably cylindrical and desirably each vessel provides a resin bed having a bed volume with a length to diameter ratio of at least about 1:1 and preferably >3:1. The vessel can be filled with a selected and preferably water-wet strongly acidic cation resin to give a depth of at least 18 in (46 cm) in the vessel. Two or more (multiple) vessels may be connected in series. Multiple vessels may also be connected in parallel to facilitate continuous operation. The advantages of multiple vessels are well known to those skilled in the art.

Channeling through the resin contained in the vessel, e.g., the resin bed can substantially reduce the effective capacity (meq/ml) of the resin. Techniques to minimize such channeling are well known to those skilled in the art.

Vessels are typically mounted vertically. Two or more vessels may be connected in series and/or in parallel or in combinations of these. Preferably the flow during the treatment of the AHA is downflow. Regeneration and flushing flows are preferably in the opposite, i.e., countercurrent direction, upflow in this preferred process. This reversed flow procedure provides a surprisingly effective demineralization at the outflow of the vessel during treatment of the AHA solution.

A filter to prevent elution of particulates can be attached to the outlet of each vessel. An example of a suitable filter is a 10-micrometer in-line filter. The vessels can also be equipped with a positive displacement pump, having no metal parts that contact the liquid, such as a digitally controlled TEFLON diaphragm pump. An example pump head is an all-TEFLON diaphragm pump head, Model No. 07090-62 (Cole-Parmer Instrument Company, Vernon Hills, Ill., USA).

Prior to use, the strongly acidic cation resin or resins is placed into the suitable vessels and the resin is optionally flushed, that is, contacted with a flow of DI water to substantially remove water-soluble materials from the resin. For example, the resin can be washed with at least 0.5 volume of the resin, and more preferably at least one volume of resin of DI water. The DI water flush produces washed resin. The washed resin is contacted with a strong acid in a desired flow direction to produce an acid-treated resin. The direction of flow of acid is preferably the same as that of the wash water, if used to previously or subsequently wash the resin. Though any strong acid can be used, it is preferred that a solution of from about 2 to about 10% sulfuric acid in DI water be used. The acid should have a low metal concentration. Suitable commercially available grades of sulfuric acid are Sulfuric Acid, VLSI, 95.0-97.0% and other such analyzed products with as low or lower metal concentration (Mallinkrodt Baker, Chesterfield, Mo., USA). Other strong mineral acids may be used instead of sulfuric, provided that grades having equivalent low metal concentrations are used.

The volume of strong acid used can depend on its concentration and the volume of the strongly acidic cation resin. General guides include (a) that it be sufficient to provide at least about 40 equivalents sulfuric acid/ft³ resin (1400 eq/m³) when preparing resin nominally already in the H⁺ form; or (b) that it provides from at least about 0.75 to at least about 2.0 equivalents of sulfuric acid/equivalent exchange capacity of the resin.

Used strongly acidic cation resin may be regenerated after step (e) using steps b and c of the procedure for resin preparation above, preferably with first contacting the resin with a flow of deionized water prior to step (b) of contacting the resin with a strong acid, to produce a washed resin and using sufficient strong mineral acid to restore the resin to its pristine low metal concentration. For regeneration of used resin that is not in the H⁺ form, the general guides include (1) that the volume of strong acid used is sufficient to provide at least about 80 equivalents sulfuric acid/ft³ resin (2800 eq/m³); or (2) that it provides from at least about 3.0 to at least about 4.0 equivalents of sulfuric acid/equivalent exchange capacity of the resin. The acid treatment is followed with DI water flushing until the resistivity of the output approaches that of fresh DI water. In practice, flushing with DI water is continued until the resistivity of the output is at least about 5 M ohm, higher resistivity may be desirable, and requires additional time and volume of DI water to be achieved.

An aqueous composition comprising an AHA having a total metal concentration and/or individual metal concentration higher than that described above for electronics grade wet chemicals, that is, greater than 1000 μg/kg of total metals and greater than 250 μg/kg of an individual metal can then be contacted with the acid-treated resin. The contact can be carried out by any means known to one skilled in the art. For example, the solution can be passed through the strongly acidic cation resin by a mechanical force such as, for example, a positive displacement pump. Because such means are well known to one skilled in the art, a description is omitted herein for the interest of brevity. The rate of the aqueous composition or solution flowing through a resin bed can be conventionally measured as the “empty bed contact time” (EBCT). The EBCT is the time for one empty bed volume of feed to pass through the bed. The empty bed volume is the volume occupied by the wet resin. The EBCT can be about at least 1 minute, preferably at least 5 minutes, more preferably at least 10 minutes, or more preferably at least 15 minutes. The shorter contact times progressively are less efficient in the use of the resin capacity. To prevent dilution of the final product from residual DI water in the bed, the first portion of glycolic acid through the beds may be collected separately as a forecut. A forecut is an initial portion of the eluate that is set aside for disposal or further treatment since it does not meet product specifications. This forecut can be taken until the concentration of the glycolic acid is such that the entire subsequent main cut meets the final specifications for glycolic acid concentration. Such forecuts typically have low metal concentration, and may be concentrated, retreated with new or regenerated resin, or used to aid the flushing of the DI water from a prepared resin bed.

The term “sample” is used to describe an aliquot, such as the 15 mL aliquot of the Examples, taken at suitable intervals for analyte measurement. The term “fraction” is used to describe the total volume of product eluted from the vessel, such as the approximately 600 mL of eluate in the Examples, collected between samples.

Samples for metals analysis (twelve metals: aluminum, calcium, chromium, copper, iron, lead, magnesium, manganese, nickel, potassium, sodium, and zinc) are taken at suitable intervals, such as hourly, with appropriate rigorous control to prevent contamination. When metal analyses reach the maximum product specifications, typically significant capacity remains in the resin. Optionally, the flow through the containers may be continued and the product, having a diminished metal concentration, but too high a metal concentration to meet final product specifications, can be collected for subsequent reprocessing with fresh or regenerated strongly acidic cation resin prepared as described hereinabove. In practice and based on the specific set of specifications to be met, fractions are collected and combined until the average concentration of metals in the combined eluates approaches one or more of the specification limits. The column may, however, continue to be used to treat feed solution, but the effluent is separated for an application having either less stringent specifications or for retreatment through freshly prepared or regenerated resin. These subsequent fractions, while not meeting specifications, nevertheless contain reduced metal concentrations versus the original feed, and thus contribute a lower metal load in the retreatment process.

Temperatures and concentrations can be controlled to prevent crystallization or precipitation of the AHA. Solubility versus temperature information for aqueous solutions of AHAs is known or easily determined. Typical operating temperatures are ambient. In the case of a 70% glycolic acid solution, for instance, manufacturer's recommendations include a recommendation for storage at temperatures between 10° to 50° C. to avoid formation of any solid phase.

Metal analyses can be made using any suitably sensitive methods, such as inductively coupled plasma mass spectrometry (ICP-MS).

Treated AHA solutions, such as those of the composition of this invention, meeting specifications are transferred to suitable non-metallic packaging containers or containers that are lined to prevent contact with metals. Suitable packaging and lining materials that may contact the low total metal concentration AHA compositions of the present invention are as described above for cation exchange resin containers and other process equipment.

The handling of ultra-low metal concentration liquids requires their rigorous protection from inadvertent contamination. These techniques are well known to those skilled in the art.

Also provided is a process for using the composition of this invention or the product produced by the process of this invention for cleaning of substrates or semiconductor-related equipment such as, for example, removing plasma ash residues or removing post etch residue. The process comprises contacting a substrate with a solution comprising the composition of this invention to clean the substrate. For purposes herein, this solution is referred to as the “cleaning solution.” By “cleaning” it is meant to remove an undesirable material, such as a residue from producing the substrate, from the substrate. The substrate can be a surface or structure of a fully or partially fabricated electronic device or processing equipment. The substrate can comprise insulating materials, non-insulating materials, and combinations thereof.

The substrate can be, for example, a surface or structure of a metal or silicon-based material. The term “metal” used herein as related to a surface or structure can include metal, metal alloy, metal compound, or combinations of two or more thereof. Examples of a metal surface or a metal structure include, but are not limited to, metal plugs, such as tungsten plugs; metal or metal compound stacks including two or more of titanium nitride, aluminum, copper, aluminum/copper alloy, titanium, tungsten, tantalum, and other metals useful in semiconductor fabrication; or at least a portion of one or more layers of metal nitrides, metal oxides, metal oxynitrides, and/or metal alloys with atoms or compounds other than metals such as phosphorus, boron, or sulfur, or combinations of two or more thereof.

Silicon-based material used here to provide a surface or structure can comprise silicon, silicon oxides, nitrides, oxynitrides, and modified silicon materials with atoms or compounds other than silicon such as phosphorus, boron, sulfur, carbon, fluorine, or germanium and combinations of two or more thereof.

The cleaning solution comprising the composition of this invention for cleaning of substrates or semiconductor-related equipment is an aqueous solution and can further comprise from about 1% to about 15%, by weight, of an organic solvent. The composition of the invention comprising low metal concentrations can be present in the cleaning solution in the range of from about 0.01% to about 30% or preferably from about 1% to about 10% by weight of the alphahydroxyacid. Typically, the cleaning solution is prepared from the composition of this invention by dilution. Preferably “super” DI water is used to dilute the composition of this invention to prepare the cleaning solution. “Super” DI water has a very high resistivity, such as about 18 M ohm or higher. More preferably, not only is “super” DI water used, but also rigorous procedures are followed to minimize contamination, such as the use of a Class 100 cleanroom environment.

The solution for treating a substrate can also comprise an acid such as phosphoric acid or salt thereof in the range of from about 0.01% to about 5%; a base as defined below in the range of from about 0.01% to about 5%; a fluorine-containing compound in the range of from about 0.001% to about 0.5%; other chelating agents in the range of from about 0.01% to about 5%; a surfactant in the range of from about 0.01% to about 1%; or combinations of two or more thereof. The acid can be phosphoric acid or its salt, pyrophosphoric acid, periodic acid, fluorosilicic acid, methanesulfonic acid, or combinations of two or more thereof. The base can be a quaternary ammonium compound, ammonium hydroxide, an alkylammonium hydroxide, hydroxylamine, alkylhydroxylamine, an alkanolamine, another amine or combinations of two or more thereof. The fluorine compound can be hydrogen fluoride, ammonium fluoride, ammonium biflouride, or combinations of two or more thereof. Other chelating agents can be catechol, ethylenediamine tetraacetic acid (EDTA), diethylene triamine pentaacetic acid (DTPA), or combinations of two or more thereof. The surfactant can be an epoxy-polyamide compound or other known surfactants. The pH of the solution can be between about 1.5 to about 10, or about 2 and about 6.

For example, a solution can comprise or consist essentially of about 1% glycolic acid, from about 1.5% to about 2.5% of phosphoric acid, from about 0.5% to about 1% of hydroxylamine, and from about 0.005% to about 0.04% of an ammonium bifluoride. An alternative solution can comprise or consist essentially of about 3% glycolic acid, from about 1.5% to about 2.5% of phosphoric acid, from about 0.5% to about 1% of hydroxylamine, from about 0.005% to about 0.04% of an ammonium bifluoride, and from about 0.05% to about 0.2% of an epoxy-polyamide compound. Still, another alternative solution can comprise or consist essentially of about 5% glycolic acid, from about 1.5% to about 2.5% of phosphoric acid; from about 0.5% to about 1% of a hydroxylamine; and from about 0.005% to about 0.1% of ammonium fluoride. All percents given for the solutions are by weight.

A cleaning solution comprising the composition disclosed above may be contacted with a semiconductor substrate by any method known to one skilled in the art such as, for example, submerging the substrate in the solution, by spraying directly onto the surface of the substrate, by flowing the solution over the substrate, or by flushing the substrate with the cleaning solution. Contact may be improved by mechanical agitation, ultrasonic and megasonic waves, bath circulation, rotation or other motion of the substrate. By improving contact, time required for cleaning and damage to the substrates may be reduced.

The contacting can be carried out under ambient pressure, at a temperature in the range of from about 0 to about 100° C., or from about 10 to about 50° C., or from about 20 to about 30° C. for a period of time, which can depend on the residue to be removed, temperature, or method of application and can be in the range of from about 1 to about 100 minutes, or from about 3 to about 50 minutes, or from about 3 to about 15 minutes, or from about 3 to about 20 minutes, or from about 5 to about 10 minutes, or from about 5 to about 15 minutes, or from about 5 to about 20 minutes. The contacting can also be ascertained by evaluating cleaning efficiency and material compatibility at various times.

The process can optionally comprise rinsing the substrate. Rinsing can be done with water, alcohol such as isopropyl alcohol, or both water and alcohol, or any rinse material known to one skilled in the art such as, for example, that disclosed in U.S. Pat. No. 5,981,454.

### Materials and Test Methods

Test Method 1. Preparation and operation of Cation Exchange Resin Columns

Deionized (DI) water used in the examples had a resistivity of 17.8 M ohm or greater, and was obtained from a Sybron-Barnstead NANOPURE II “turn-key” unit, available from Barnstead-Thermolyne (Dubuque, Iowa, USA).

In each Example, fresh cation exchange resin was charged to a 2.5 cm diameter×100 cm borosilicate glass column to a depth of approximately 24″ (61 cm). The resin was then flushed (downflow) with DI water until the effluent resistivity was at least 10 M ohm. The resin was then treated (upflow) at 10 mL/min with approximately 2 bed volumes of 4% sulfuric acid (electronics grade), and subsequently flushed (upflow) with DI water until the bulk acid was displaced, as determined by effluent density. The bed was then further flushed (downflow) with DI water until the effluent resistivity read at least 5 M ohm. The alphahydroxyacid solutions to be purified were stored and fed under nitrogen. The alphahydroxyacid solutions were then fed downflow (counter-current to the acid pre-treatment step) at 10 mL/min through the pre-conditioned column. Generally, 15-mL samples of the bed effluent were taken every hour (about every 600 mL of eluate) into polyethylene bottles that had been triple-rinsed with DI water, and analyzed for metals using ICP-MS (Test Method 2).

Test Method 2. Determination of Microgram/Kilogram Concentrations of Metals in Tartaric and Glycolic Acid Solutions Using Inductively Couipled Plasma-Mass Spectrometry.

The samples of tartaric and glycolic acids, taken as described in Test Method 1, were diluted with DI water by a factor of 10 and analyzed by inductively coupled plasma-mass spectrometry (ICP-MS). All sample preparation and analyses were carried out in a Class 100 cleanroom environment. Determination limits for each element were approximately 1 μg/kg (1 part per billion, ppb) in the solution as collected from the ion exchange resin column. For analyses close to a detection limit, some sample-to-sample variation is not unexpected.

The equipment used included an Agilent 7500s or 7500cs ICP-MS system with a ShieldTorch interface (Agilent Technologies, Palo Alto Calif.); ChemStation and FileView software packages (Agilent Technologies, see above); a ASX-100 Micro Volume Autosampler (Agilent Technologies, see above); a Mettler AG285-CR analytical balance (Mettler-Toledo, Columbus Ohio); a Biohit e1000 electronic pipettor (Biohit Oyj, Helsinki, Finland); 1-mL polypropylene pipet tips (Corning, Inc., Corning N.Y.); 15-mL and 50-mL polypropylene centrifuge tubes with screw caps (Corning, Inc., see above); 18-M ohm deionized water (ASTM Type II water, ASTM D1193); high purity argon (stock number ARG-240L, MG Industries, Malvern Pa.); high purity hydrogen (scientific grade, MG Industries, see above); 100-μL PFA TEFLON® micronebulizer (stock number PFA-100, Elemental Scientific, Omaha NB); quartz torch with 2.5 mm ID (stock number G1833-65423, Agilent Technologies, see above); 6-mL autosampler vials (stock number G13160-65303 (Agilent Technologies, see above); ICP-MS tuning solution with Li, Y, Ce, Tl and Co at 10 μg/kg each (stock number 5184-3566, Agilent Technologies, see above); DUPEX CAL 3A multielement standard customized and supplied by Inorganic Ventures (Lakewood N.J.) that contains 27 elements at 100 mg/kg each and includes the twelve elements of interest (Na, Mg, Al, K, Ca, Cr, Mn, Fe, Ni, Cu, Zn, and Pb); ULTREX II 65.0-70% ultrapure nitric acid (J. T. Baker, Phillipsburg N.J.); OMNITRACE 69.0-70.0% nitric acid (EMD Chemicals, Gibbstown N.J.) for cleaning purposes only); General-purpose plastic container with cover and at least 5-L capacity (VWR Scientific, West Chester Pa.); and unused, sealable plastic bags (VWR Scientific, see above).

Tubes and vials did not necessarily have to be prepared in a cleanroom environment. The general-purpose container was prepared by filling it with DI water and adding OMNITRACE nitric acid to make an approximate 5% solution. Screw caps were removed from sample tubes and the tubes and caps submerged with minimal air pockets along with the autosampler vials into the nitric acid bath. The container was covered and the items allowed to leach in the bath for a minimum of 16 h, when they were removed from the bath, rinsed thoroughly with DI water, and sealed and stored in plastic bags until ready for use.

Working standard solutions were prepared by pipetting 500 microL (μL) of the 100 mg/kg CAL3A stock standard into a clean 50-mL tube. DI water was used to dilute to the 50-mL mark, and the solution designated as the “1000 μg/kg working standard”, which therefore contained 1000 μg/kg of each of the 12 analytes. 500 μL of the 1000 μg/kg working standard were pipetted into a clean 50-mL tube, and diluted to the 50-mL mark with DI water. This solution is designated as the “10 μg/kg working standard”.

Calibration standards were prepared by selecting one sample from the sample batch in order to prepare matrix-matched standards. This sample was designated as Sample A and is not a feed sample. 1.0 mL of Sample A was pipetted into each of nine clean 15-mL centrifuge tubes.

The following amounts of the working standards were pipetted into the tubes as shown in Table 1 below, and each tube was then diluted to the 10-mL mark with DI water. Each tube was kept capped with a clean screw cap until analysis.

TABLE 1
Preparation of matrix-matched calibration standards

| Calibration | Volume, mL | | | | Elemental |
|---|---|---|---|---|---|
| Standard | Sample | | 1000 μ/kg | | Concn. |
| (μg/kg) | A | 10 μg/kg std. | std. | Total | (μg/kg) |
| 0 | 1.0 | 0 | 0 | 10.0 | 0 |
| 0.1 | 1.0 | 0.1 | 0 | 10.0 | 0.1 |
| 0.5 | 1.0 | 0.5 | 0 | 10.0 | 0.5 |
| 1 | 1.0 | 1.0 | 0 | 10.0 | 1 |
| 2 | 1.0 | 2.0 | 0 | 10.0 | 2 |
| 10 | 1.0 | 0 | 0.1 | 10.0 | 10 |
| 50 | 1.0 | 0 | 0.5 | 10.0 | 50 |
| 100 | 1.0 | 0 | 1.0 | 10.0 | 100 |
| 200 | 1.0 | 0 | 2.0 | 10.0 | 200 |

Tartaric and glycolic acid solutions were prepared by taring a clean, empty 15-mL centrifuge tube on the analytical balance, pipetting 1 mL of an eluate sample as received into the tube and recording the exact weight. This sample was diluted to a total of 10 mL and the exact weight again recorded. The tube was kept capped with a clean screw cap until analysis. This procedure was repeated for each sample and the dilution factor for each sample calculated by dividing the total weight of the diluted sample by the weight of the sample as received.

In the case of feed samples having much higher cation concentrations, an additional dilution step was performed. The above procedure was repeated using 1 mL of the previously diluted feed sample, which was diluted to 10 mL using the same weighing and dilution factor calculation. The tube was kept capped with a clean screw cap until analysis. This procedure was repeated for all feed samples and the overall dilution factor recalculated for each. As a result of this additional dilution, feed samples were analyzed approximately 10 times more dilute than eluate samples. For feed samples with a concentration of 32,000 μg/kg or higher for a particular analyte, see below.

Unless noted otherwise, all analyses were performed on an Agilent 7500s (or 7500cs) ICP-MS system with ShieldTorch, using “cool plasma” (low RF power) conditions. Typical parameters for the argon plasma under cool plasma conditions are shown in Table 2. Carrier and blend gasses were derived from the same source of argon. The quartz torch as described above was used.

TABLE 2
Typical argon plasma parameters for cool plasma

| Parameter | Cool Plasma Conditions |
|---|---|
| RF power (W) | 600-900 |
| Sampling depth (mm) | 11-13 |
| Torch-H (mm)* | −2 to +2 |
| Torch-V (mm)* | −2 to +2 |
| Carrier gas (L/min) | 0.8-1.3 |
| Blend gas (L/min) | 0.0-0.4 |
| Spray chamber temp (° C.) | 2 |

*relative horizontal (H) and vertical (V) position of the torch to the mass spectrometer.

A self-aspirating 100-microL PFA micronebulizer was used to introduce a sample or standard into the instrument's spray chamber at an approximate flow rate of 100 μL/min. The ICP-MS was tuned while introducing the tuning solution (see above) and following the guidelines in the Agilent 7500 ICP-MS ChemStation Operator's Manual (stock No. G18333-65423, July 2001, Agilent Technologies, see above). Typically, torch and lens parameters were optimized to maximize the signal for Co (mass/charge ratio or m/z 59) while minimizing the signals that were indicative of plasma and instrumental interferences (i.e., m/z 40, m/z 56, or m/z 80).

In instances when the background signal of the argon plasma at m/z 40 is relatively high, measurements of calcium were made with the use of the reaction cell of an Agilent 7500cs ICP-MS system. With reaction cell, normal plasma conditions as outlined in Table 3 were utilized. Hydrogen served as the reaction gas at a flow rate of 2.7 mL/min. Reference is made to the Agilent 7500 ICP-MS ChemStation Operator's Manual (see above) was used for additional details on the use and tuning with the reaction cell. The reaction cell procedure is further discussed in the “Appendix” below.

TABLE 3
Typical normal plasma parameters for use with the reaction cell

| Parameter | Normal Plasma Conditions |
|---|---|
| RF power (W) | 1200-1600 |
| Sampling depth (mm) | 4-10 |
| Torch-H (mm)* | −2 to +2 |
| Torch-V (mm)* | −2 to +2 |
| Carrier gas (L/min) | 0.8-1.3 |
| Makeup gas (L/min) | 0-0.4 |
| Spray chamber temp (° C.) | 2 |

*See footnotes for Table 2.

A program, referred to as a method by the ChemStation software, controlled the measurement and data acquisition for each sample and calibration standard. Analytes and pertinent parameters for each are given in Table 4.

TABLE 4
ChemStation measurement parameters

| | | | | Total |
|---|---|---|---|---|
| | | Measurement | | Acquisition |
| | Mass | Time per | Number of | Time per |
| Analyte | (m/z) | Mass (s)* | Repetitions | Mass (s)* |
| Na | 23 | 0.5 | 3 | 1.5 |
| Mg | 24 | 0.5 | 3 | 1.5 |
| Al | 27 | 0.5 | 3 | 1.5 |
| K | 39 | 0.5 | 3 | 1.5 |
| Ca | 40 | 0.5 | 3 | 1.5 |
| Cr | 52 | 0.5 | 3 | 1.5 |
| Mn | 55 | 0.5 | 3 | 1.5 |
| Fe | 56 | 0.5 | 3 | 1.5 |
| Ni | 60 | 0.5 | 3 | 1.5 |
| Cu | 63 | 0.5 | 3 | 1.5 |
| Zn | 67 | 0.5 | 3 | 1.5 |
| Pb | 208 | 0.5 | 3 | 1.5 |

*These columns refer to the time period (s) during which the mass spectrometer collects data at the mass listed in Column 2 before jumping to the next mass.

Sample uptake and stabilization times were set accordingly in the same ChemStation method and were optimized during the tuning process since these times can vary between individual nebulizers. These times were adjusted so that the signal counts had a maximum relative standard deviation of 5% (n=200) when data acquisition began.

Two post-measurement sample rinses of at least 20 seconds (s) each were set in the ChemStation method. The two rinses consisted of two separate vials of deionized water with approximately 3% ultrapure nitric acid.

A different and more abbreviated ChemStation method was used when measuring calcium by an Agilent 7500cs (“Appendix” below). This program was similar to the first with the major exceptions that normal plasma conditions were used and calcium was the only analyte measured.

For the initial calibration verification, an aliquot of each of the nine calibration standards (Table 1) was placed into clean autosampler vials and the vials were loaded into the autosampler of the ICP-MS system. The standards were analyzed sequentially from the one with the lowest spiked concentrations (0 (zero) μg/kg) to the one with the highest (200 μg/kg). After all of the standards were analyzed and using the ChemStation software, the calibration curve for each analyte was verified to be linear with an r²-value of at least 0.95.

Analyses of calibration standards and samples were made by placing an aliquot of each sample into clean autosampler vials, which were loaded into the autosampler of the ICP-MS system. A ChemStation sequence list was set up, saved, and executed. All sample dilution factors calculated above were entered in the sequence list. Note that the Agilent 7500cs utilized an additional sequence employing a similar yet different ChemStation method when measuring calcium (see Appendix below).

All nine calibration standards were analyzed at the beginning of the sequence and again at the end of sequence to verify that no significant signal drift occurred over the course of the measurements. All nine calibration standards are preferentially run periodically throughout the sequence, for instance each time after approximately 12 samples have been analyzed. At least one instrument blank consisting of DI water with approximately 10% ultrapure nitric acid was included in the sequence.

Feed samples, with higher analyte concentrations, were analyzed last in the sequence to minimize any possible cross-contamination between samples.

Results from the calibration standards were used to generate two separate calibration curves in ChemStation's Data Analysis module. The “low concentration” calibration curve utilized standards 0 μg/kg, 0.1 μg/kg, 0.5 μg/kg, 1 μg/kg, 2 μg/kg, and, in some instances, 10 μg/kg. The “high concentration” calibration curve utilized standards 0 μg/kg, 10 μg/kg, 50 μg/kg, 100 μg/kg, and 200 μg/kg. Each curve was initially constructed by the method of standard additions using Sample A (see above). Each curve was then converted to an external standard calibration curve using the ChemStation software.

All samples were processed using the low concentration curve and the “Do List” command. The calculated results (now multiplied by the appropriate dilution factors) were then compiled in the FileView software where they were saved in a spreadsheet format. This previous step was repeated using the high concentration curve to produce a second set of results in a spreadsheet format.

Results generated from FileView were compiled and formatted in a spreadsheet, with the majority of results derived from the low concentration curve. Only measurements that occurred above the range of the low concentration curve were reported from the high concentration curve. For example, if the low concentration curve for a particular measurement range was 0-2 μg/kg, any measurements above 16 μg/kg (2 μg/kg multiplied by a typical dilution factor of approximately 8) were taken from the data of the high concentration curve. Consequently feed sample results were derived from the high concentration curve. Additionally, any measurement greater than an approximate value of 32,000 μg/kg (2×200 μg/kg×a typical dilution factor of 80) required an additional dilution and reanalysis of the sample. High levels of calcium in the glycolic acid Examples (Tables 7-9) necessitated this reanalysis.

All results for the final reporting were rounded to the nearest whole pg/kg unit (i.e., 4.7 μg/kg reported as 5 μg/kg) and reported with a maximum of three significant figures. Measurements of 1 μg/kg or <1 μg/kg were reported as the worst case value of 1 μg/kg to enable averaging (see Examples below).

“Appendix” on the Measurement of Calcium.

The use of an argon plasma under normal plasma conditions made calcium difficult to measure by ICP-MS since the primary isotopes of both calcium and argon had an atomic mass of 40 amu. ICP-MS analysts in recent years have rectified this problem by reducing the power of the plasma (referred to as “cool plasma” conditions), which in turn reduced the amount of interfering argon ions relative to those of calcium. This approach was utilized when using an Agilent 7500s ICP-MS system.

The upgraded Agilent 7500cs instrument was not necessarily optimized for cool plasma conditions because it had the ability to minimize or eliminate the effect of plasma interferences by another approach. The 7500cs contained a reaction cell between the plasma and the mass analyzer. In the case of calcium measurements, hydrogen was introduced into the cell, and the interfering argon ions could be eliminated by the one of two reactions:
Ar⁺+H₂→Ar+H₂⁺(1)
or
Ar⁺+H₂→ArH⁺+H (2)

In the case of the charge transfer reaction (1), the resulting Ar was no longer ionized and detected by the mass spectrometer at m/z 40. As a result of the atom abstraction (2), Ar⁺ became ArH⁺, which now could only be detected at m/z 41. Ca⁺ did not react with H₂ within the reaction cell; thus, as a result of Reactions 1 and 2, only Ca⁺ could be detected and measured at m/z 40. This was the approach utilized by this method to measure calcium when using an Agilent 7500cs ICP-MS.

Note that another concern regarding calcium measurements by ICP-MS is that calcium is one of the most common contaminants from sample handling, solvents, and other sources of background contamination. This analytical method required matrix-matched standards and utilized the method of standard additions, which together could mask background contamination at ultratrace levels especially when a true blank of calcium-free glycolic acid is neither known nor available. This emphasized the need for the use of a cleanroom environment and ultratrace techniques when performing this method.

The reference for the above ICP-MS procedures is the Agilent 7500 ICP-MS ChemStation Operator's Manual (stock No. G18333-65423, July 2001, Agilent Technologies, Palo Alto Calif.).

### EXAMPLES

Feed solutions of AHA were stored and fed to the resin columns under a nitrogen atmosphere. In the Examples, 15-mL samples of column eluate were collected hourly (Test Method 1) and individually analyzed for twelve metals (aluminum, calcium, chromium, copper, iron, lead, magnesium, manganese, nickel, potassium, sodium, and zinc) using Test Method 2. The eluate or fraction between each 15-mL analytical sample was approximately 600 mL. Analyte concentration in a given fraction is taken as the analyte concentration found in the sample taken at the end of the fraction. In the tabulated results, the averaged concentrations are shown, corresponding to the concentration that would be obtained by combining groups of fractions. In each Example, the first fraction (the forecut, see discussion above) includes water flushed from the prepared and water-washed column. As a result, this forecut has a concentration of the AHA that is significantly below the AHA in the feed solution. This first fraction was rejected, although in practice it could be concentrated and combined with treated but non-specification eluate for retreatment. The next several fractions were identified as Factions 1 to 19 or 1 to 21 in each run, depending on the number of fractions collected. In practice, eluate fractions meeting set specifications would be combined to provide a product with averaged concentration. Fraction 1 and subsequent fractions contain essentially the feed concentration of AHA. The tables show the metal analyses for the feed, Fractions 1-5 (total volume 3 L), Fractions 1-10 (total volume 6 L), Fractions 1-15 (total volume 9 L), and either Fractions 1-20 (total volume 12 L) or Fractions 1-19 (total volume 11.4 L). The concentrations of this last group of fractions depended on the number of fractions collected. Each group of fractions also shows the total analyte concentration.

All fittings and process tubing were either PFA or TEFLON to avoid metal contamination. A TEFLON diaphragm pump drove flow through the bed.

The densities of the 50% tartaric acid and 70% glycolic acid feeds are 1.26 g/cc and 1.24 g/cc respectively, thus the weight of the 600-mL fractions are 756 g and 744 g for tartaric acid and glycolic acids respectively. The concentration of AHA in each fraction is 378 g for tartaric acid and 521 g for glycolic acid.

Examples 1 and 2 were purification tests using a prepared 50 wt % L-(+)-tartaric acid solutions. Example 1 used DOWEX MONOSHERE M-31 cation resin, Example 2 used DOWEX MONOSPHERE 650C cation resin. Both resins are strongly cationic exchange resins.

Examples 3-5 were purification tests using a commercial 70% glycolic acid (70% Tech Grade Glycolic Acid, from E.I. du Pont de Nemours and Company, Wilmington Del.) as the feed. Example 3 used DOWEX MONOSHERE M-31 resin, Example 4 used DOWEX MONOSHERE 650C resin, and Example 5 used a 50-50 vol % layered bed of DOWEX MONOSHERE M-31 and 650C resins.

For Examples 3-5 (glycolic acid), two system changes were made after running Examples 1 and 2 (tartaric acid). First, a valve was installed in the effluent line to isolate the sampling valve from the downstream process lines to prevent any backflow of product that may have contacted metal surfaces in the mass flow sensor used during sampling. Secondly, the beds were drained of water to within approximately 1″ (2.5 cm) above the resin beds prior to the AHA feed, to help sharpen the transition period between water and full-strength acid.

Additionally, Example 5 also incorporated a pre-treatment of the glycolic acid solution feed in which the feed solution was dosed with excess ferrous sulfate to attempt reduction of hexavalent chromium to the Cr(III) from. Cr(VI) present in aqueous solution as chromate or dichromate anions would not be amenable to cation resin removal. Iron (II) sulfate heptahydrate, (68 mg, analytical grade) was added to 16.98 kg of 70% aqueous glycolic acid solution and stirred under nitrogen at room temperature overnight.

TABLE 5
Fraction Analyses for Example 1

| | |
|---|---|
| Feed: | 50 wt % Tartaric acid |
| Resin: | DOWEX MONOSPHERE M-31 |
| Treatment Rate: | 10 ml/min |
| Bed Dimensions: | 2.5 cm diameter × 59.7 cm |

| Example 1 | Grouped Fraction Average Concentration, μg/kg | | | | | |
|---|---|---|---|---|---|---|
| Metal | Feed | 1-5 | 1-10 | 1-15 | 1-20 | Minimum* |
| Na | 7080 | 179 | 325 | 2091 | 3414 | 163 |
| Mg | 1595 | 3 | 2 | 2 | 2 | 1 |
| Al | 8935 | 9 | 9 | 8 | 8 | 6 |
| K | 5645 | 68 | 67 | 322 | 1383 | 63 |
| Ca | 4120 | 176 | 119 | 119 | 108 | 54 |
| Cr | 283 | 133 | 170 | 191 | 204 | 102 |
| Mn | 4 | 2 | 2 | 2 | 2 | 1 |
| Fe | 249 | 15 | 12 | 12 | 11 | 6 |
| Ni | 151 | 143 | 145 | 147 | 147 | 137 |
| Cu | 9 | 1 | 1 | 1 | 1 | 1 |
| Zn | 87 | 32 | 27 | 29 | 28 | 20 |
| Pb | 1 | 1 | 1 | 1 | 1 | 1 |
| Total | 28159 | 761 | 880 | 2923 | 5308 | 665 (a) |

Fractions collected: 21, included above: 20
*Minimum concentration for a specific analyte measured in any Fraction. For the “Total” row, the minimum value (a) is the minimum total analyte value in any Fraction, not the sum of the minimum values.

Table 5 shows total metal concentration was reduced to less than 1000 μg/kg and individual metal concentrations were reduced to less than 200 μg/kg.

TABLE 6
Fraction Analyses for Example 2

| | |
|---|---|
| Feed: | 50 wt % Tartaric acid |
| Resin: | DOWEX MONOSPHERE 650C |
| Treatment Rate: | 10 ml/min |
| Bed Dimensions: | 2.5 cm diameter × 61.0 cm |

| | Grouped Fraction Average | | | | | |
|---|---|---|---|---|---|---|
| Example | Concentration, μg/kg | | | | | |
| 2 | | | | | | Mini- |
| Metal | Feed | 1-5 | 1-10 | 1-15 | 1-19 | mum* |
| Na | 4275 | 64 | 854 | 2504 | 3275 | 35 |
| Mg | 1290 | 16 | 18 (b) | 16 (b) | 16 (b) | 2 |
| Al | 19 | 31 | 27 | 28 | 31 | 5 |
| K | 3930 | 37 | 250 | 1032 | 1572 | 5 |
| Ca | 2265 | 93 | 84 | 76 | 72 | 5 |
| Cr | 824 | 209 | 253 | 288 | 319 | 176 |
| Mn | 15 | 5 | 6 | 5 | 6 | 2 |
| Fe | 586 | 57 | 58 | 56 | 52 | 5 |
| Ni | 12 | 6 | 6 (b) | 6 (b) | 6 (b) | 5 |
| Cu | 13 | 5 | 5 (b) | 5 (b) | 7 (b) | 5 |
| Zn | 82 | 7 | 7 | 6 | 6 | 3 |
| Pb | 5 | 5 | 5 | 5 | 5 | 5 |
| Total | 13316 | 535 | 1573 | 4028 | 5367 | 371 (a) |
| Fractions collected and included above: 19 | | | | | | |
| *and (a) See definition below Table 5. | | | | | | |
| (b) Analyses for Example 2 Fraction 9 for Mg, Ni, and Cu were anomalous and very high in comparison with all other samples. Statistical analysis using the American Society for Testing Materials (ASTM) “Standard Practice for Dealing with Outlying Observations” (ASTM E178-80, reapproved in 1989) indicated these specific analyses | | | | | | |
| # (Fraction 9, Mg 699 μg/kg, Ni 279 μg/kg, and Cu 799 μg/kg) were outliers by this statistical test. Skewness and kurtosis plots confirmed this conclusion. Consequently, for the calculation of averages, | | | | | | |
| # the three-outlier values were replaced with the average of the corresponding analyses for the preceding and following fractions (Fractions 8 and 10). | | | | | | |

Table 6 shows total metal concentration was reduced to less than 1000 μg/kg and individual metal concentrations were reduced to less than 250 μg/kg. The DOWEX MONOSHPERE 650 resin removes sodium more efficiently than DOWEX MONOSPHERE M-31 initially.

TABLE 7
Fraction Analyses for Example 3

| | |
|---|---|
| Feed: | 70 wt % Glycolic Acid |
| Resin: | Dowex Monosphere M-31 |
| Treatment Rate: | 10 ml/min |
| Bed Dimensions: | 2.5 cm diameter × 59.7 cm |

| Example 3 | Grouped Fraction Average Concentration, μg/kg | | | | | |
|---|---|---|---|---|---|---|
| Metal | Feed | 1-5 | 1-10 | 1-15 | 1-20 | Minimum* |
| Na | 28800 | 53 | 54 | 53 | 59 | 51 |
| Mg | 12350 | 1 | 2 | 1 | 2 | 1 |
| Al | 2575 | 5 | 4 | 4 | 3 | 2 |
| K | 2635 | 16 | 15 | 15 | 16 | 14 |
| Ca | 37950 | 24 | 26 | 21 | 22 | 6 |
| Cr | 551 | 42 | 43 | 42 | 42 | 36 |
| Mn | 78 | 1 | 1 | 1 | 1 | 1 |
| Fe | 3710 | 1 | 2 | 2 | 2 | 1 |
| Ni | 1805 | 2 | 3 | 3 | 3 | 2 |
| Cu | 408 | 2 | 2 | 2 | 3 | 1 |
| Zn | 987 | 1 | 1 | 1 | 1 | 1 |
| Pb | 5 | 1 | 1 | 1 | 1 | 1 |
| Total | 91854 | 149 | 154 | 146 | 153 | 127 (a) |

Fractions collected and included above: 20
*and (a) See definition below Table 5.

Table 7 shows significantly better metal removal for glycolic acid as feed than was obtained with tartaric acid (Examples 1 and 2 in Table 5 and 6). Total metal concentration was less than 200 μg/kg for all fractions, and individual metal concentrations, except for sodium, were less than 50 μg/kg.

TABLE 8
Fraction Analyses for Example 4

| | |
|---|---|
| Feed: | 70 wt % Glycolic Acid |
| Resin: | Dowex Monosphere 650C |
| Treatment Rate: | 10 ml/min |
| Bed Dimensions: | 2.5 cm diameter × 59.7 cm |

| Example 4 | Grouped Fraction Average Concentration, μg/kg | | | | | |
|---|---|---|---|---|---|---|
| Metal | Feed | 1-5 | 1-10 | 1-15 | 1-19 | Minimum* |
| Na | 30350 | 5 | 10 | 14 | 27 | 3 |
| Mg | 12200 | 1 | 1 | 1 | 1 | 1 |
| Al | 2660 | 5 | 5 | 4 | 4 | 2 |
| K | 2820 | 44 | 86 | 95 | 99 | 21 |
| Ca | 38250 | 8 | 8 | 10 | 13 | 5 |
| Cr | 415 | 34 | 38 | 36 | 35 | 31 |
| Mn | 66 | 1 | 1 | 1 | 1 | 1 |
| Fe | 2715 | 9 | 13 | 10 | 9 | 5 |
| Ni | 1145 | 3 | 3 | 3 | 3 | 2 |
| Cu | 266 | 3 | 3 | 3 | 3 | 2 |
| Zn | 141 | 1 | 1 | 1 | 1 | 1 |
| Pb | 5 | 1 | 1 | 1 | 1 | 1 |
| Total | 91033 | 114 | 169 | 179 | 197 | 93 (a) |

Fractions collected and included above: 19
*and (a) See definition below Table 5.

Table 8 shows total metal concentration was less than 200 μg/kg for all fractions. Individual metal concentrations were less than 50 μg/kg initially, and, except for potassium, were less than 50 μg/kg for all fractions. The DOWEX MONOSHPERE 650 resin clearly removes sodium more efficiently than DOWEX MONOSPHERE M-31, although with slightly poorer performance for potassium.

TABLE 9
Fraction Analyses for Example 5

| | |
|---|---|
| Feed: | 70 wt % Glycolic Acid |
| Resin: | Dowex Monosphere 650C (bottom); |
| | Dowex Monosphere M-31 (top) |
| Treatment Rate: | 10 ml/min |
| Bed Dimensions: | 2.5 cm diameter × 30.0 cm (top layer); |
| | 2.5 cm diameter × 30.7 cm (bottom layer) |
| Note: | Feed was pre-treated with FeSO₄.7H₂O. |

| Example 5 | Grouped Fraction Average Concentration, μg/kg | | | | | |
|---|---|---|---|---|---|---|
| Metal | Feed | 1-5 | 1-10 | 1-15 | 1-20 | Minimum* |
| Na | 27500 | 8 | 12 | 14 | 21 | 2 |
| Mg | 10900 | 1 | 1 | 5 | 4 | 1 |
| Al | 2540 | 4 | 4 | 4 | 4 | 1 |
| K | 3805 | 28 | 48 | 59 | 64 | 4 |
| Ca | 35250 | 16 | 16 | 17 | 19 | 7 |
| Cr | 399 | 20 | 24 | 25 | 26 | 4 |
| Mn | 70 | 1 | 1 | 1 | 1 | 1 |
| Fe | 1940 | 11 | 12 | 12 | 12 | 9 |
| Ni | 1170 | 5 | 4 | 4 | 4 | 1 |
| Cu | 276 | 2 | 3 | 2 | 2 | 1 |
| Zn | 1217 | 6 | 8 | 8 | 7 | 1 |
| Pb | 5 | 1 | 1 | 1 | 1 | 1 |
| Total | 85072 | 103 | 135 | 153 | 165 | 37 (a) |

Fractions collected and included above: 20
*and (a) See definition below Table 5.

Table 9 shows total metal concentration at less than 200 μg/kg for all grouped fractions, and individual metal concentrations at less than 50 μg/kg for all grouped fractions. Example 5 has significantly better removal of Cr in all grouped fractions compared with Examples 3 and 4, due to pretreatment with a reducing agent (ferrous sulfate heptahydrate, 68 mg in 16.98 kg of 70% aqueous glycolic acid, to reduce Cr(VI) to Cr(III) in the feed prior to treatment.

Tables 7, 8, and 9 show that for Examples 3 and 4, for glycolic acid, DOWEX MONOSPHERE M-31 resin is effective at reducing potassium (to below 20 μg/kg) but is less effective with sodium. Conversely, the DOWEX MONOSPHERE 650C was less effective at removing potassium and more effective at removing sodium. In Example 5, both resins were present and the combination shows improved sodium removal compared with Example 3 and improved potassium compared with Example 4.

1. A composition comprising an alphahydroxyacid and one or more metals wherein the total metal concentration is less than 1000 μg/kg and the concentration of any individual metal of the composition is less than 250 μg/kg.

2. The composition of claim 1 wherein the metal is selected from the group consisting of aluminum, calcium, chromium, copper, iron, lead, magnesium, manganese, nickel, potassium, sodium, and zinc, and combinations of two or more thereof.

3. The composition of claim 2 wherein the composition is in the form of an aqueous solution.

4. The composition of claim 3 wherein the total metal concentration is less than 500 μg/kg and the concentration any individual metal of the composition is less than 150 μg/kg.

5. The composition of claim 4 wherein the total metal concentration is less than about 200 μg/kg.

6. The composition of claim 5 wherein the total metal concentration is less than about 100 μg/kg.

7. The composition of claim 4 wherein the individual metal concentration is less than about 100 μg/kg.

8. The composition of claim 7 wherein the individual metal concentration is less than about 50 μg/kg.

9. The composition of claim 8 wherein the total metal concentration is less than about 25 μg/kg.

10. The composition of claim 1 wherein the metal is selected from the group consisting of sodium, magnesium, aluminum, potassium, calcium, iron, nickel and zinc and combinations of two or more thereof having a concentrations of sodium, magnesium, aluminum, potassium, calcium, iron, nickel, and zinc of less than 200 μg/g of the composition.

11. The composition of claim 10 having a concentrations of sodium, magnesium, aluminum, potassium, calcium, iron, nickel, and zinc of less than 100 μg/g of the composition.

12. The composition of claim 1 wherein the alphahydroxyacid is selected from the group consisting of glycolic acid, lactic acid, tartaric acid, and citric acid.

13. The composition of claim 12 wherein the alphahydroxyacid is glycolic acid or tartaric acid.

14. The composition of claim 13 wherein the alphahydroxyacid is glycolic acid.

15. The composition of claim 3 wherein the concentration of alphahydroxyacid is 50 to 99% of the solubility limit of the acid in the composition.

16. The composition of claim 15 wherein the concentration of alphahydroxyacid is 75 to 98% of the solubility limit of the acid in the composition.

17. A process to produce an alphahydroxyacid with ultra-low metal concentration comprising the steps of:
(a) providing one or more vessels comprising therein at least one strongly acidic cation resin;
(b) contacting the resin with a flow of a strong acid to produce an acid-treated resin;
(c) washing the resin with a flow in a concurrent flow direction to the flow of strong acid of deionized water to produce a resin substantially free of soluble acid;
(d) contacting the acid-treated and washed resin with a flow in a countercurrent flow direction to the flow of strong acid of a feed composition comprising an alphahydroxyacid and one or more metals wherein the total metal concentration is greater than about 1000 μg/kg and the individual metal concentration is greater than about 250 μg/kg to produce a resin-treated alphahydroxyacid composition and spent resin; and
(e) separating and recovering the resin-treated alphahydroxyacid composition.

18. The process of claim 17 further comprising keeping the alphahydroxyacid feed composition under a blanket of an inert gas.

19. The process of claim 18 further comprising contacting the resin with a flow of deionized water prior to step (b) of contacting the resin with a strong acid, to produce a washed resin.

20. The process of claim 19 wherein the flow direction of strong acid is upflow and the flow direction of the alphahydroxyacid feed composition is downflow.

21. The process of claim 20 further comprising in a step prior to providing one or more vessels comprising therein a cation resin, a step of treating the feed composition to reduce Cr(VI) compounds to Cr(III) compounds.

22. The process of claim 21 wherein the reducing step comprises contacting the feed composition with a reductant Wherein the reductant is selected from the group consisting of a solution comprising a soluble reducing agent or a gaseous reductant, such as sulfur dioxide (which forms sulfurous acid in solution).

23. The process of claim 22 wherein the soluble reducing agent is selected from the group consisting of a ferrous salt, hydrogen peroxide, potassium iodide, and sodium sulfite.

24. The process of claim 20 further comprising regenerating the spent resin for reuse after step (e), wherein the process comprises (a′) contacting the resin with a flow of deionized water to produce a washed resin; (b′) contacting the washed resin with a flow of a strong acid to produce an acid-treated resin; and (c′) washing the acid-treated resin with a flow in a concurrent flow direction to the flow of strong acid of deionized water to produce a resin substantially free of soluble acid.

25. A process for cleaning comprising contacting a substrate with a solution comprising a composition comprising an alphahydroxyacid and one or more metals wherein the total metal concentration is less than 1000 μg/kg and the concentration of any individual metal of the composition is less than 250 μg/kg.

26. The process of claim 25 wherein the substrate is a surface or structure of a fully or partially fabricated electronic device or processing equipment.

27. The process of claim 25 wherein the substrate is a surface or structure of a metal or silicon-based material.

28. The process of claim 27 wherein the substrate is a metal surface or structure.

29. The process of claim 28 wherein the substrate is metal plugs, metal or metal compound stacks, or at least a portion of one or more layers of metal nitrides, metal oxides, metal oxynitrides, metal alloys with atoms or compounds other than metals such as phosphorus, boron, or sulfur, or combinations of two or more thereof.

30. The process of claim 27 wherein the substrate is a silicon-based material surface or structure.

31. The process of claim 30 wherein the substrate is a surface or structure comprising silicon, silicon oxides, nitrides, oxynitrides, modified silicon materials with atoms or compounds other than silicon such as phosphorus, boron, sulfur, carbon, fluorine, or germanium, and combinations of two or more thereof.

32. The process of claim 25 wherein the solution comprises from about 1% to about 15%, by weight, of an organic solvent.

33. The process of claim 25 wherein the solution further comprises an acid, a base, a fluorine-containing compound, other chelating agents or combinations or two or more thereof.

34. The process of claim 33 wherein the acid is phosphoric acid, the base is hydroxylamine, the fluorine-containing compound is ammonium bifluoride and the other chelating agent is an epoxy-polyamide compound.
